(19) United States
(12) Patent Application Publication
(10) Pub. No.: US 2006/0191076 A1
(43) Pub. Date: Aug. 31, 2006

(54) Method of treating a textile using a colour changing form

(76) Inventor: Marcio Henrique Perissinotto Bonfa, Sao Paulo (BR)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(21) Appl. No.: 10/547,596

(22) PCT Filed: Feb. 19, 2004
(86) PCT No.: PCT/EP04/01609

§ 371 Date: Sep. 1, 2005

(30) Foreign Application Priority Data
Mar. 5, 2003 (GB) 0305010.1

(51) Int. Cl. A01H 5/02

(52) U.S. Cl. 8/148; 8/158

(57) ABSTRACT

The present invention provides a visual cue to the consumer. The visual cue is for a cleaning method, whereby a textile is treated with a liquid detergent in the form of a foam and wherin the visual cue is a colour change to said foam.

### FIELD OF INVENTION

The present invention provides a consumer cue, permitting association of a colour change with a function of the product.

### BACKGROUND OF THE INVENTION

Branding is a multifaceted concept. Branding is public perception, history of a product, and association with the company who makes and markets the product. Branding is the product's name, its visual identity, its packaging, and the consumer's association with the product and product quality, style, or functionality. Branding is the trust and greater identification that consumers have in one product over another. Consumers also make judgements about brands based on learned notions.

A consumer will associate a perception with an effect even though in many cases the perception and effect are unrelated. An example of an unrelated perception with effect is for example the use of mint in toothpaste. The mint does not effect cleaning of the teeth but the consumer associates the taste with clean teeth.

It is known to use a robust dye in detergent powders, for example as found in OMO™ Progress as sold in Brazil. The dye used imparts a blue colour both to the detergent composition and an aqueous wash medium to which the detergent composition added. The dye used maintains its integrity in the detergent composition as formulated and is unaffected upon addition to an aqueous medium.

U.S. Pat. No. 5,929,004 discloses a detergent for cleaning tire wheels, which is applied in a foamy state onto the tire wheels, thereby allowing a grime adhered to the surface of the tire wheels to come off from the tire wheels. The detergent contains a surfactant, at least one alkali compound selected from the group consisting of ammonia and amino group-containing alkali compounds, and a colour change indicator capable of changing its own colour when transferred from an alkaline condition to a neutral condition.

### SUMMARY OF THE INVENTION

The present invention provides a colour cue, the cue being all the more noticeable by the consumer because the colour appears when the product is added to an aqueous medium or exposed to the atmosphere.

The colour cue of the present invention is provided by a pH dependent chromophore; the colour of the pH dependent chromophore is dependent upon the acidity or the alkalinity of its environment.

The present invention provides a method of treating a textile, the textile carrying a stain, the method comprising the following steps: (i) applying a liquid detergent composition in the form of a foam to a textile, the liquid detergent composition comprising: a base, a detergent, a pH dependent chromophore, and carriers and adjuncts to 100%; (ii) waiting until the foam, as applied, has changed colour from its originally applied colour; (iii) rinsing the portion of the textile to which the foam has been applied with water; and, (iv) drying the textile.

Further the method may include the step of dissolving a detergent concentrate in water to provide the liquid detergent composition for application. This detergent concentrate may be selected from the group consisting of: liquid and granular solid detergent concentrates.

Many detergent products are formulated to a high pH and the change in colour may serve to indicate that the detergent product is functioning at the optimum pH. The colour change has other performance benefits. The alkalinity of the composition may be tuned so that the colour changes at an preordained time. A colour change may also indicate that the textile has been treated for sufficient time. An initial colour also provides improved stain to treatment registration such that an environmental benefit is obtained because less detergent is used in cleaning a stain.

### DETAILED DESCRIPTION OF THE INVENTION

The pH Dependent Chromophore

Many pH dependent chromophores are commonly referred to as indicators. However, it is not essential that the pH dependent chromophore is reversible in its colour change.

In this regard, pH dependent chromophores other than an indicator may be used. Below is found a Table in which examples of various pH dependent chromophores (indicators) are found. There are many standard texts available that give lists of pH dependent chromophores (indicators).

| | Colour | Colour | | |
|---|---|---|---|---|
| Indicator | Acid | Base | pKln | pH range |
| Thymol Blue - | red | yellow | 1.5 | 1.2-2.8 |
| 1st change | | | | |
| Methyl Orange | red | yellow | 3.7 | 3.2-4.4 |
| Bromocresol | yellow | blue | 4.7 | 3.8-5.4 |
| Green | | | | |
| Methyl Red | yellow | red | 5.1 | 4.8-6.0 |
| Bromothymol Blue | yellow | blue | 7.0 | 6.0-7.6 |
| Phenol Red | yellow | red | 7.9 | 6.8-8.4 |
| Cresol Red | yellow | red | 8.2 | 7.2-8.8 |
| Metacresol | yellow | purple | 8.32 | 7.4-9.0 |
| purple | | | | |
| Thymol Blue - | yellow | blue | 8.9 | 8.0-9.6 |
| 2nd change | | | | |
| Phenolphthalein | colourless | Pink | 9.4 | 8.2-10.0 |
| Thymolphthalein | colourless | blue | 10.0 | 9.3-10.5 |

A mixture of pH dependent chromophores/indicators may be used in the present invention.

The amount of the pH dependent chromophore/indicator present in the composition will vary depending on the extinction coefficient/colour intensity of the indicator required. The amount of pH dependent chromophore having a UV-vis spectrum that changes with pH in the range 1 to 14 required is that sufficient for a discernable change in colour to be observable by the human eye. Suitably, the pH dependent chromophores, or mixtures thereof, are present in the detergent composition in the range 0.0001 to 0.5 wt %, preferably 0.001 to 0.4 wt %, most preferably 0.002 to 0.3 wt %.

Foam Dispensed pH Dependent Cue

The present invention is used as a liquid format where the composition is dispensed as foam and the dispensed product interacts with the environment. Foam has a high surface area to volume ratio and rapidly absorbs air from the atmosphere resulting in a pH change of the foam. If the foam is alkaline the pH of the foam decreases as carbon dioxide is absorbed from the atmosphere. When the appropriate pH dependent chromophore is present a colour change is observed providing a visual colour change. In this case the dispenser of the foam provides the pH changing means by dispensing the foam to a carbon dioxide containing environment (the atmosphere). The bulk within the foam dispenser may or may not be in contact with atmospheric gasses. Nevertheless, it is preferred that the internal gaseous environment does not readily exchange with the external gaseous environment when the dispenser is not in use. Suitable and preferred pH dependent chromophores are, for example, phenolphthalein and thymol blue. Suitable dispensing devices for the foam are found in EP 03250593.5 and EP 03250595.0.

Anionic Surfactant, Non-ionic surfactant and Amphoteric Surfactants or mixtures thereof may be used in this foam aspect of the present invention. Examples of suitable surfactants systems that may be used for the foam are as follows:

sodium lauryl alkyl sulphate (LAS), sodium lauryl ether sulphate (SLES) and coco amidopropyl betaine (CAPB); sodium lauryl alkyl sulphate (LAS) non-ionic (NI) and coco amidopropyl betaine (CAPB); primary alcohol sulphate (PAS), sodium lauryl ether sulphate (SLES) and coco amidopropyl betaine (CAPB); primary alcohol sulphate (PAS), sodium lauryl ether sulphate (SLES) and non-ionic (NI); and sodium lauryl alkyl sulphate (LAS) and non-ionic (NI).

The Detergent Composition

The composition preferably comprises a surfactant and optionally other conventional detergent ingredients. It is preferred that the composition used to make the foam comprises 0.001% wt/wt to 20% wt/wt of a detergent(s). It is most preferred that the detergent composition is alkaline. The alkalinity may be provided by, for example, sodium hydroxide, sodium carbonate, sodium silicate, an amine, sodium tripolyphosphate (STP) and/or zeolite. This alkalinity is preferably provided for by sodium carbonate and/or sodium hydroxide. The invention in its second aspect provides an enzymatic detergent composition which comprises from 0.1-50% by weight, based on the total detergent composition, of one or more surfactants. This surfactant system may in turn comprise 0-95% by weight of one or more anionic surfactants and 5 to 100% by weight of one or more nonionic surfactants. The surfactant system may additionally contain amphoteric or zwitterionic detergent compounds, but this in not normally desired owing to their relatively high cost. The enzymatic detergent composition according to the invention will generally be used as a dilution in water of about 0.05 to 2%.

In general, the nonionic and anionic surfactants of the surfactant system may be chosen from the surfactants described “Surface Active Agents” Vol. 1, by Schwartz & Perry, Interscience 1949, Vol. 2 by Schwartz, Perry & Berch, Interscience 1958, in the current edition of “McCutcheon's Emulsifiers and Detergents” published by Manufacturing Confectioners Company or in “Tenside-Taschenbuch”, H. Stache, 2nd Edn., Carl Hauser Verlag, 1981.

Suitable nonionic detergent compounds which may be used include, in particular, the reaction products of compounds having a hydrophobic group and a reactive hydrogen atom, for example, aliphatic alcohols, acids, amides or alkyl phenols with alkylene oxides, especially ethylene oxide either alone or with propylene oxide. Specific nonionic detergent compounds are C₆-C₂₂ alkyl phenol-ethylene oxide condensates, generally 5 to 25 EO, i.e. 5 to 25 units of ethylene oxide per molecule, and the condensation products of aliphatic C₈-C₁₈ primary or secondary linear or branched alcohols with ethylene oxide, generally 5 to 40 EO.

Suitable anionic detergent compounds which may be used are usually water-soluble alkali metal salts of organic sulphates and sulphonates having alkyl radicals containing from about 8 to about 22 carbon atoms, the term alkyl being used to include the alkyl portion of higher acyl radicals. Examples of suitable synthetic anionic detergent compounds are sodium and potassium alkyl sulphates, especially those obtained by sulphating higher C₈-C₁₈ alcohols, produced for example from tallow or coconut oil, sodium and potassium alkyl C₉-C₂₀ benzene sulphonates, particularly sodium linear secondary alkyl C₁₀-C₁₅ benzene sulphonates; and sodium alkyl glyceryl ether sulphates, especially those ethers of the higher alcohols derived from tallow or coconut oil and synthetic alcohols derived from petroleum. The preferred anionic detergent compounds are sodium C₁-C₁₅ alkyl benzene sulphonates and sodium C₁₂-C₁₈ alkyl sulphates. Also applicable are surfactants such as those described in EP-A-328 177 (Unilever), which show resistance to salting-out, the alkyl polyglycoside surfactants described in EP-A-070 074, and alkyl monoglycosides.

Preferred surfactant systems are mixtures of anionic with nonionic detergent active materials, in particular the groups and examples of anionic and nonionic surfactants pointed out in EP-A-346 995 (Unilever). Especially preferred is surfactant system that is a mixture of an alkali metal salt of a C₁₆-C₁₈ primary alcohol sulphate together with a C₁₂-C₁₅ primary alcohol 3-7 EO ethoxylate.

The nonionic detergent is preferably present in amounts greater than 10%, e.g. 25-90% by weight of the surfactant system. Anionic surfactants can be present for example in amounts in the range from about 5% to about 40% by weight of the surfactant system.

The detergent composition may take any suitable physical form, such as a powder, granular composition, tablets, a paste or an anhydrous gel.

Bleaching Species

The present invention may be used in combination with bleaching species. These species may be, for example: a) a peroxygen bleach species alone and/or in combination with a bleach activator and/or a transition metal catalyst; and 2) a transition metal catalysts in a composition substantially devoid of peroxygen species.

The use of bleaching catalysts for stain removal has been developed over recent years and may be used in the present invention. Examples of transition metal catalysts that may be used are found, for example, in: WO0148298, WO0060045, WO0248301, WO0029537 and WO0012667. The catalyst may alternatively be provided as the free ligand that forms a complex in situ. Bleach activators are well known in the art, for example TAED, and SNOBS. Peroxygen bleaching agents are also well known in the art, for example, peracids (e.g., PAP), perborates, percarbonates, peroxyhydrates, and mixtures thereof. Specific preferred examples include: sodium perborate, commercially available in the form of mono- and tetra-hydrates, and sodium carbonate peroxyhydrate. Other examples of peroxyl species and activators are found in WO02077145 as well as other transition metal catalyst.

Other Adjuncts

Builders, polymers and other enzymes as optional ingredients may also be present in the detergent composition as found in WO0060045 and WO0034427.

### Foam Examples

Table 1 gives formulations A to J that when made up to 100% with water provide suitable foaming compositions.

TABLE 1

| | Formulation | | | | | | | |
|---|---|---|---|---|---|---|---|---|
| | A | B | C | D | F | G | I | J |
| | % | % | % | % | % | % | % | % |
| LAS | 1.5 | 1.5 | 2 | 2 | 2.5 | 5 | 3 | 1.5 |
| NaOH | 0.204 | 0.204 | 0.272 | 0.272 | 0.34 | 0.68 | 0.476 | 0.204 |
| Non Ionic | 1.5 | 1.5 | 2 | 2 | 2.5 | — | — | — |
| (Unitol L70) | | | | | | | | |
| LESS | — | — | — | — | — | — | 1 | 1.5 |
| Formaldehyde | 0.08 | 0.08 | 0.08 | 0.08 | 0.08 | 0.08 | 0.08 | 0.08 |
| phenolphtha- | 0.002 | 0.002 | 0.002 | 0.002 | 0.002 | 0.002 | 0.002 | 0.002 |
| lein | | | | | | | | |
| Perfume | 0.15 | 0.15 | 0.15 | 0.15 | 0.15 | 0.15 | 0.15 | 0.15 |
| EDTA | 0.3 | 0.3 | 0.3 | 0.3 | 0.3 | 0.3 | 0.3 | 0.3 |

1. A method of treating a textile, the textile carrying a stain, the method comprising the following steps:
(i) applying a liquid detergent composition in the form of a foam to a textile, the liquid detergent composition comprising: a base, a detergent, a pH dependent chromophore, and carriers and adjuncts to 100%;
(ii) waiting until the foam, as applied, has changed colour from its originally applied colour;
(iii) rinsing the portion of the textile to which the foam has been applied with water; and,
(iv) drying the textile.

2. A method of treating a textile according to claim 1, wherein the base is selected from sodium hydroxide and sodium carbonate.

3. A method of treating a textile according to claim 1, wherein the UV-vis spectrum of the pH dependent chromophore changes with pH in the range 1 to 14.

4. A method of treating a textile according to claim 3, wherein the UV-vis spectrum of the pH dependent chromophore changes with pH in the range 7.5 to 10.5.

5. A method of treating a textile according to claim 1, wherein the pH dependent chromophore is selected from the group consisting of: Phenolphthalein; thymolphthalein; Thymol Blue; Methyl Orange; Bromocresol Green; Methyl Red; Bromothymol Blue; and, Phenol Red.

6. A method of treating a textile according to claim 1, wherein the pH dependent chromophore is present in the composition in the range 0.0001 to 0.5 wt %.

7. A method of treating a textile according to claim 1, wherein the method comprises the step of dissolving a detergent concentrate in water to provide the liquid detergent composition for application.

8. A method of treating a textile according to claim 7, wherein the detergent concentrate is selected from the group consisting of: liquid and granular solid detergent concentrates.
